(19) United States
(12) Patent Application Publication
(10) Pub. No.: US 2014/0039549 A1
(43) Pub. Date: Feb. 6, 2014

(54) TISSUE CLOSURE DEVICE AND METHOD OF DELIVER AND USES THEREOF

(71) Applicants: Ziv Belsky, Haifa (IL); Gilad Hizkiyahu, Givat-Ela (IL); Gil Bolotin, Zikhron Yakov (IL)

(72) Inventors: Ziv Belsky, Haifa (IL); Gilad Hizkiyahu, Givat-Ela (IL); Gil Bolotin, Zikhron Yakov (IL)

(73) Assignee: Novogate Medical LTD, Migdal HaEmek (IL)

(21) Appl. No.: 14/113,095

(22) PCT Filed: Apr. 23, 2012
(86) PCT No.: PCT/IB12/52033

§ 371 Date: Oct. 21, 2013

Related U.S. Application Data
(60) Provisional application No. 61/477,638, filed on Apr. 21, 2011.

(51) Int. Cl. A61B 17/12

(52) U.S. Cl. CPC A61B 17/12013
USPC 606/215

(57) ABSTRACT

The present invention relates generally to a tissue closure device and in particular to a tissue closure device having a plurality of configurations providing for delivery and use in minimally invasive procedures. A tissue closure device (100) is provided from shape memory or super-elastic materials for closing a tissue site opening (50), the device comprising a plurality of tissue anchors (130) extending from a closed loop surface (110) wherein the plane of said tissue anchors is provided at an angle essentially perpendicular with respect to the plane of said closed loop surface; a. wherein said plurality of tissue anchors (130) comprise a proximal end (130p) and a distal end (130d); said distal end provided for anchoring said device onto tissue (50); and wherein said proximal end (130p) is fluid with and extends from said closed loop surface (110); and b. said closed loop surface (110) having a plurality of configurations including: a delivery configuration (1OOd), an open configuration (1000) and a closed configuration (100c) wherein said closed loop surface (110) may undergo a transformation between one of said plurality of configurations to another; said delivery configuration (1OOd) defining a low profile of said device (100) and adapted for delivery to a tissue site (50) through a small profile access point having a first diameter and wherein said closure device is utilized to close a tissue site having a second diameter such that said second diameter is larger than said first diameter.

### FIELD OF THE INVENTION

The present invention relates generally to a tissue closure device and in particular to a tissue closure device having a plurality of configurations providing for delivery and use in minimally invasive procedures.

### BACKGROUND OF THE INVENTION

Advancement in medicine and in particular minimally invasive procedures and surgery, such as keyhole surgery, require the formation of various puncture sites to define access points to the internal anatomy. Such procedures require the use of various customized tools, port, trocars or cannula to gain access to the required anatomy with minimal incision.

The requirement of such access points depends on the type of procedure being performed and may occur in varying locations. For example, angioplasty, keyhole hernia repair, gallbladder removal, require a number of topical access points through the skin to gain access to the abdominal cavity. Other procedures, such as transapical delivery of medical devices to the left or right ventricle for example during valve correction and/or replacement, require access through specific anatomical structures such as the ribs and a beating heart.

Many of the low profile procedures and tools, such as angioplasty, stents and guidewires, have been made possible due to the development of shape memory alloys and materials for use in medical applications.

Shape memory alloys are a group of materials that, after being deformed, return to a predetermined shape when heated. This memory effect is caused by a temperature-dependent crystal structure of the material. One-way shape memory alloys remember a single shape, to which they return upon being heated. Two-way shape memory alloys remember two different shapes, the first at a relatively low temperature, and the second at a higher temperature.

Various material types such as organic, plastics, metallic, are capable of employing shape memory and are well known in the art. An article made of such materials can be deformed from an original, heat-stable configuration, to a second, heat-unstable configuration. The article is said to have shape memory for the reason that, upon the application of a trigger, for example heat, it can be caused to revert, or to attempt to revert, form its heat-unstable configuration to its original, heat-stable configuration, i.e. it “remembers” its original heat-stable configuration or shape.

Many medical devices employ shape memory alloys and materials, for their super-elastic and/or plastic properties, that allow a device to have more than one stable configuration, usually a low profile configuration and an operative and/or functional configuration. For example tissue closure devices, sutures, and staples integrate and/or utilize memory shape alloys. Following minimally invasive procedures, closure of the access points, incisions and intervening tissue is generally required. This requirement has led to the development of various types of tissue closure devices, such as sutures, staples, surgical tapes, and tissue adhesives. While the most prevalent wound closure method is the suture using needles and sutures to close a wound, however, the process of suturing is time consuming in particular during minimally invasive procedures where there isn't much room to perform the suture.

When suturing a tissue incision that was used as a keyhole access point, the most prevalent closure method utilized is a “purse-string” suture. A purse string suture is formed around a tissue site (hole), where a single thread is stitched to surround the hole and then pulled tight emulating a purse-string to close the hole. However, performing this suture requires time, skill, practice and requires sufficient access to the wound site to allow closure of the hole. Typically, the purse-string suture is applied as soon as the cannula is inserted into the body.

State of the art staples have other drawbacks of only holding the tissue together at certain points, which does not take advantage of the entire tissue surface area to create a strong bond, potentially leading to leakage.

### SUMMARY OF THE INVENTION

There is an unmet need for, and it would be highly useful to have, a tissue closure device provided to close a tissue opening wherein the tissue closure device may be delivered to a tissue site through a small access point so as to close a tissue site (native anatomical opening, incision, wound), with a closure device capable of exerting sufficient radial force in a concentric fashion about the tissue site.

The present invention overcomes the deficiencies of the background by providing a device for tissue closure configured to have a concentric arrangement that provides a stable and efficient radial closing force about a tissue opening, and in particular to such a tissue closure device configured to be delivered through an access point having a first (access point) diameter and utilized to close a tissue site/opening having a second diameter, such that the second diameter is larger than the first diameter, while allowing sufficient margins about the tissue site for the closure device to associate with the tissue and allowing closure of the tissue with sufficient radial force. Most preferably, the tissue closure device of the present invention may be delivered through an aperture having a smaller diameter than the diameter of the tissue site that the closure device securely closes.

Most preferably the tissue closure device, may assume a delivery and/or storage and/or low profile configuration adapted to be delivered to a tissue site through an access point of a small diameter, for example a catheter, trocar or the like, once through to the tissue site the device expands to assume an open configuration having a diameter larger than the diameter of the access point, and thereafter the closure device contracts and/or closes to assume a closed configuration so as to close the tissue site.

FIG. 1A-B show prior art tissue closure devices, the drawback of the prior art tissue closure devices is that their mode of delivery is a direct mode of delivery where the size of the tissue site to be closed is limited by the size of the delivery canal, while the closing forces applied to close the tissue site are limited.

For example, FIG. 1A shows tissue closure device, described in US Patent Publication No. US2012/0022586 to Whitman et al., where a delivery device is associated with the tissue closure device comprising a plurality of tissue anchors about the perimeter of the delivery device. The device depicted in FIG. 1A cannot close a tissue opening that is larger than the diameter of the delivery tool itself and therefore is limited in its ability to close an internal tissue opening, having a diameter larger than the diameter of the access point or delivery canal or deployment tool.

FIG. 1B shows a different tissue closure device, described in US Patent Publication No. 2007/0198057 to Gelbart et al, here again the tissue closure device is limited to closing a tissue openings that is the same size or smaller than the diameter of the delivery tool itself.

Accordingly, prior art devices, as shown, do not provide a tissue closure device capable of closing a tissue site that has a diameter, that is larger than the diameter of the access point itself. Instead prior art devices require a one to one relationship between the diameter of the access point and the diameter of the tissue opening that is to be closed. Prior art devices are limited to such a one to one ratio because the closure device cannot assume a low profile configuration sufficient to securely close a tissue opening that is substantially larger than the access point and/or delivery canal while applying sufficient and substantial closing force to close the tissue, for example a myocardium.

Within the context of this application the terms tissue site opening may be interchangeably used with the terms tissue site, tissue opening, wound, hole, incision, opening through tissue, or the like term as a term of art. Optionally a tissue site opening may be disposed on any tissue and/or tissue surface, including external, topical tissue such as the skin, and/or internal tissue and/or anatomical structures and/or anatomical opening.

Within the context of this application the terms surgical closure device and tissue closure device may be used interchangeably to refer to an optional embodiment of the present invention defining a device for closing a tissue site opening, hole, wound, incision, opening through tissue, whether disposed on any tissue type including external, topical tissue such as the skin, or internal tissue or anatomical structures such as myocardium.

Within the context of this application the term access point and access point tools may be interchangeably refer to a cannula, port, catheter, trocar, keyhole or the like.

Within the context of this application the terms continuous loop and closed loop surface may be used interchangeably to refer to a ring like structure forming a portion of the tissue closure device according to an optional embodiment of the present invention, utilized for closing wound and/or tissue opening.

Within the context of this application the term holding force refers to the force exerted by tissue anchors on the tissue that it is anchored and/or embedded in and exerted when a force perpendicular to the tissue is applied on the device and the tissue anchors. Most preferably the holding force prevents the closure device of the present invention from being extracted from the tissue site by a perpendicular force that may be exerted on it.

Within the context of this application the term closing force refers to the force required to close the tissue wound, lesion, opening, hole or the like. The term closing force may be interchangeably used with the term radial force to refer to forces applied by the device on the tissue or through the device on the tissue to close the tissue lesion, wound, opening, hole toward the center of the tissue lesion and/or wound, and/or opening and/or hole.

Within the context of this application the term transition and transformation may be interchangeably used to refer to the change from one configuration of the tissue closure device or any portion thereof, from one configuration to another. For example, a changed from a closed configuration to an open configuration may be collectively referred to herewith as a transition or transformation. Any reference to such transition and/or transformation between configurations throughout this application may be optionally be provided by manual manipulation with a dedicated tool and/or device or alternatively may be a triggered response of the device itself, as a response to an environmental change. Optionally an environmental change may for example include but is not limited to temperature change, application of heat, application of cold, electric current, electrical field, electromagnetic field, magnetic field, chemical solution, the like or any combination thereof, that may provide for changing one configuration to another configuration.

Within the context of this application the terms adapted and/or customized may refer to change, adaptation, reconfiguration, customization, alone or in any combination thereof to control the material, material properties, sizes, dimension, thickness, or the like material properties.

Within the context of this application the terms memory shape materials and/or polymers and/or alloys, nitinol and/or super-elastic materials may be used interchangeably to refer to as materials capable of employing, shape memory where the materials may be deformed from an original, heat-stable configuration, to a second, heat-unstable configuration. The article is said to have shape memory for the reason that, upon the application of a trigger, for example heat, it can be caused to revert, or to attempt to revert, form its heat-unstable configuration to its heat-stable configuration therein it “remembers” its original heat-stable configuration or shape.

An optional embodiment of the present invention overcomes the deficiencies of the background art by providing a tissue closure device that may be anchored with a plurality of anchors and wherein closure is achieved without folding the tissue anchors within the tissue to achieve closure, while providing sufficient radial force to close a tissue site.

An optional embodiment of the present invention provides a tissue closure device including a closed loop surface having a plurality of tissue anchors seamlessly extending from the closed loop surface. Most preferably the tissue closure device may be configured to have a plurality of transitional configurations. Most preferably the closed loop surface may be configured to have at least three transitional configurations. Most preferably tissue anchors may be configured to have at least two transitional configurations.

Optionally and preferably the device according to the present invention may be provided from super-elastic materials (‘SE’) and/or memory shape polymers, shape memory alloys (‘SMA’), plastics or alloys and materials capable of super-elastic and/or memory shape properties and having a plurality of stable configurations. Such materials and/or alloys or polymers thereof may for example comprise but is not limited to Ni—Ti, Ni—Ti alloys, nitinol, Cu based alloys, Cu—Zn—Al, Au—Cd, Ni—Al, stainless steel 316, polymers, BeCu alloy, CoCr alloy, Ag—Cd, Au—Cd, Cu—Al—Ni, Cu—Sn, Cu—Zn, Cu—Zn—Si, Cu—Zn—Sn, Fe—Pt, Mn—Cu, Fe—Mn—Si, Pt alloys, Co—Ni—Al, Co—Ni—Ga, Ni—Fe—Ga, Ti—Pd, Ni—Ti—Nb, Ni—Mn—Ga, the like as is known in the art and/or any combination thereof.

Optionally and preferably the tissue closure device according to an optional embodiment of the present invention is configured to have a thickness for example from about 0.25 mm to about 2.0 mm, optionally from about 0.5 mm to about 1.75 mm, optionally and preferably from about 0.7 mm to about 1.6 mm, and most preferably about 1.5 mm.

Optionally the closed loop surface may be constructed from at least two closed loop surfaced configured in concentric manner. Optionally the tissue closure device may be configured to have a first closed loop surface, for example an outer closed ring structure, and an internal ring loop. Optionally at least two closed loop concentric structures may be combined to form the tissue closure device according to the present invention.

Optionally and most preferably tissue anchors are disposed essentially orthogonally with respect to the closed loop surface.

Optionally and preferably the tissue anchors may be provided with a thickness of about 0.5 mm. Optionally tissue anchors are provided with a length of about 5 mm to about 7 mm. Optionally and preferably tissue anchors are provide with a length of about 6 mm.

Optionally the tissue anchors may be shaped in an arrow-head like and/or barb configuration, about the distal end of the anchors. Preferably tissue anchor shape provides for securely anchoring and/or embedding tissue closure device within a given tissue. Tissue anchor distal end provided in the form of an arrow-head may be configured to have a width of from about 0.8 mm to about 3 mm, more preferably about 2 mm.

Optionally tissue anchors may comprise at least one or more skyved side projection that may extend from the sides of anchors' body. Most preferably at least one or more skyved projection may provide for easy insertion into a tissue site while preventing removal from the tissue site, as the skyved projection protrudes with a removal attempt. Optionally and most preferably skyved projections provide a holding force for the tissue anchor; therein preventing extraction from the tissue site. Optionally and most preferably skyved projections extend from the body of the anchor with the exertion of a perpendicular force on the anchor. Optionally and preferably skyved tissue anchor's may be provided from SMA or SA for example including NiTi or the like alloys.

An optional tissue anchor distal end provided in the form of an arrowhead may be configured to have an internal angle, optionally internal angle is an obtuse angle, for example from about 100 degrees to about 125 degrees, more preferably form about 110 to about 120, and most preferably about 115 degrees.

An optional tissue anchor distal end provided in the form of an arrowhead may be configured to have an external angle, optionally the external angle is an acute angel, for example from about 40 degrees to about 75 degrees, optionally and preferably from about 45 to about 65, more preferably form about 50 to about 60, and most preferably about 56 degrees.

An optional tissue anchor distal end provided in the form of an arrowhead may be configured to have an Optionally tissue anchors having a arrow-head configuration wherein the internal angle formed between the tissue anchor's central shaft and arrow-head having a diameter of about 0.15 mm.

Optionally and preferably each segments forming the closed loop surface portion of tissue closure device according to the present invention is provided with a margins of about 0.7 mm. Optionally and preferably a 0.7 mm buffer provides for maintain material properties such as flexibility.

Most preferably tissue anchors are disposed essentially perpendicular with respect to closed loop structure.

Optionally and preferably the tissue closure device is configured to have a low profile transformation temperature of between −18° C. to 25° C. (degrees Celsius) and more preferably between 10° C. to 25° C. (degrees Celsius).

An optional embodiment of the present invention comprises a tissue closure device provided from shape memory or super-elastic materials for closing a tissue site opening, the device comprising a plurality of tissue anchors extending from a closed loop surface wherein the plane of the tissue anchors is provided at an angle essentially perpendicular with respect to the plane of the closed loop surface;
wherein the plurality of tissue anchors comprise a proximal end and a distal end; the distal end provided for anchoring and/or embedding the device onto tissue; and wherein the proximal end is fluid with and extends from the closed loop surface; and
the closed loop surface having a plurality of configurations including: a delivery configuration, an open configuration and a closed configuration wherein the closed loop surface may undergo a transformation between one of the plurality of configurations to another; the delivery configuration defining a low profile of the device and adapted for delivery to a tissue site through a small profile access point having a first diameter and wherein the closure device is utilized to close a tissue site having a second diameter such that the second diameter is larger than the first diameter.

Optionally the plurality of tissue anchors comprise a plurality of configurations including: a delivery configuration and at least one functional and/or operative configuration.

Optionally the open configuration is provided with a diameter from about 16 mm to about 25 mm.

Optionally the closed configuration is provided with a concentric arrangement comprising an external zone and at least one internal zone.

Optionally the external zone is provided with a diameter from about 13 mm to about 19 mm.

Optionally the at least one internal zone is provided with a diameter from about 7 mm to about 13 mm.

Optionally the closure device may be configured to generate a closing force between the external zone and the internal zone, the closing force is configured to close the tissue site opening by exerting a radial force from about 200 g to about 500 g.

Optionally the closure device may be configured to apply a closing force gradient centered about the tissue site opening, wherein the closing force is highest nearest to the center of the tissue site opening and lowest at the periphery of the tissue site opening.

Optionally the tissue anchors adapt to a low profile formation the delivery configuration. Optionally tissue anchors may assume the low profile formation by folding about the proximal end.

Optionally the tissue anchor's level of the holding force may be adapted by manipulating or configuring the distal end of the tissue anchors.

Optionally the delivery configuration of the closure device may provide for delivery facilitated by a low profile delivery device or tool. Optionally the delivery tool may be provided in the form of a delivery catheter.

Optionally the open configuration defines a closure device central opening that may provide for performing minimally invasive surgical manipulation therethrough.

Optionally the tissue anchors' distal end may define anchoring projection configured for tissue anchoring and providing an embedding force, the projection selected from the group consisting of barb, hook, multi-barb, serration, threading, anchor, multi-prong anchor, arrow head, skyved projection anchors.

An optional embodiment of the present invention provides a method for closing a tissue site opening with a tissue closure device of the present invention, the method comprising:

associating the tissue closure device with a delivery tool or applicator;

transforming the tissue closure device to a delivery configuration;

delivering the closure device in the delivery configuration to a target tissue opening to be closed;

transforming the tissue closure device from the delivery configuration to an functional open configuration, therein defining a closure device central opening sufficient to encompass and close the targeted tissue site opening;

anchoring the tissue closure device about target tissue site opening with the aid of the delivery tool; and

allowing the closure device to complete the transformation from the functional open configuration to a functional closed configuration therein closing the tissue site opening.

Optionally delivering the tissue closure device may comprise maneuvering the closure device in its delivery configuration, through a small profile access point selected from the group consisting of trocar, cannula, keyhole, catheter, or the like.

Unless otherwise defined, all technical and scientific terms used herein have the same meaning as commonly understood by one of ordinary skill in the art to which this invention belongs. The materials, methods, and examples provided herein are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A-B are a schematic illustration of prior art tissue closure devices;

FIGS. 2A-C are schematic illustrations showing different configurations of an optional tissue anchors and tissue closure device according to an optional embodiment of the present invention; FIG. 2A shows an optional folded and/or low profile delivery configuration, with a delivery tool, FIG. 2B shows an open configuration with a delivery tool, and FIG. 2C shows the closed configuration without the delivery tool;

FIG. 3 is a flowchart depicting a method for tissue closure according to an optional embodiment of the present invention;

FIGS. 4A-E are schematic illustrations depicting an optional method according to an optional embodiment of the present invention, utilizing the tissue closure device according to the present invention, showing the low profile delivery, placement and use during a minimally invasive transapical surgical procedure; FIGS. 4D-E, shows a schematic illustration of an optional tissue closure device according to an optional embodiment of the present invention within the tissue in a closed formation showing the concentric arrangement of tissue anchors in accordance with an optional embodiment of the present invention.

FIGS. 5A-B are schematic illustrations of top view of an optional tissue closure device according to the present invention, shown in the closed configuration;

FIGS. 6A-C are schematic illustrations of a top view of optional closed loop surface of a tissue closure device according to the present invention, FIG. 6A showing an optional open configuration, FIGS. 6B-C show an optional closed configuration; and

FIGS. 7A-E are schematic illustrations of a perspective view of optional tissue anchors of tissue closure device according to an optional embodiment of the present invention.

### DESCRIPTION OF THE PREFERRED EMBODIMENTS

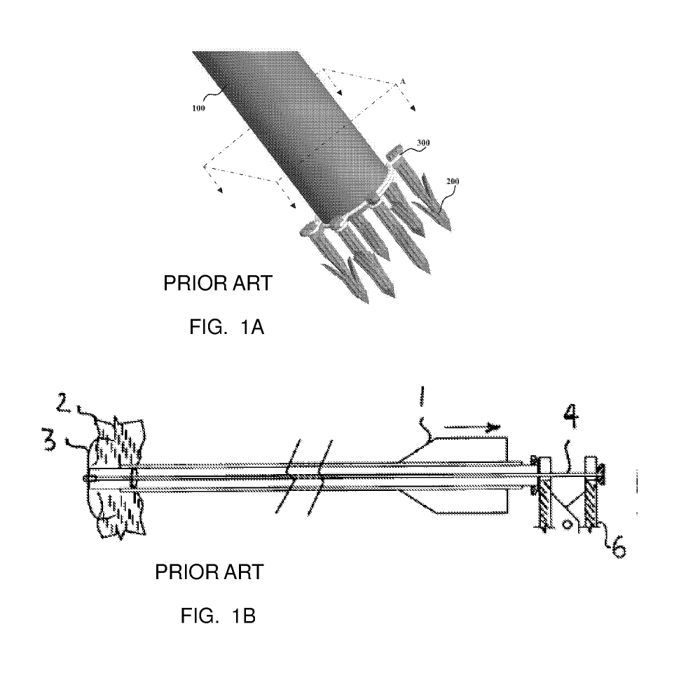

The present invention overcomes the deficiencies of the background an example of which is shown in FIG. 1A-B, by providing a device for tissue closure configured to have a concentric arrangement that provides a stable and efficient radial closing force about a tissue opening, and in particular to such a tissue closure device configured to be delivered through an access point and utilized to close a tissue opening having a diameter larger than the diameter of the access point itself. Most preferably the tissue closure device, assumes a delivery and/or low profile configuration adapted to be delivered to a tissue site through an access point, at the tissue site the device expands to assume an open configuration having diameter larger than the diameter of the access point, wherein the tissue closure device is associate with the tissue opening and thereafter contracts to assume a closed configuration so as to close the tissue site.

Optional embodiments of the present invention provide a tissue closure device, provided to close a tissue site and/or tissue opening, incision, wound, anatomy or hole, by applying a radial force and/or closing force, in a concentric fashion about the tissue opening and/or wound. Such a concentric configuration about a tissue site provides stable and efficient closing force about a tissue opening.

Optionally and preferably the concentric configuration may further provide for a force gradient across the tissue site where the highest closing force is applied closest to the center of the tissue site, while the closing force applied reduces farther away from the center of the tissue site.

The principles and operation of the present invention may be better understood with reference to the drawings and the accompanying description. The following figure reference labels are used throughout the description to refer to similarly functioning components are used throughout the specification hereinbelow.
50 targeted tissue site/opening;
100 tissue closure device;
100c tissue closure device closed configuration;
100d tissue closure device delivery configuration;
100o tissue closure device open configuration;
110 closed loop surface;
110c closed loop surface closed configuration;
110d closed loop surface delivery configuration;
110o closed loop surface open configuration;
120 internal zone surface segment
122 peripheral zone surface segment
130 tissue anchors;
130a internal angle;
130b external angle;
130c radius;
130d tissue anchor distal portion;
130e external tissue anchors;
130i internal tissue anchors;
130p tissue anchor proximal portion;
130s tissue anchor skyved projection;
200 delivery device/tool;
202 delivery device base/closed loop portion;
204 delivery device shaft/handle portion;
210 delivery catheter;
212 trocar;
220 surgical tool;
R1,R2,R3 concentric radius; and
R0 closure device opening.

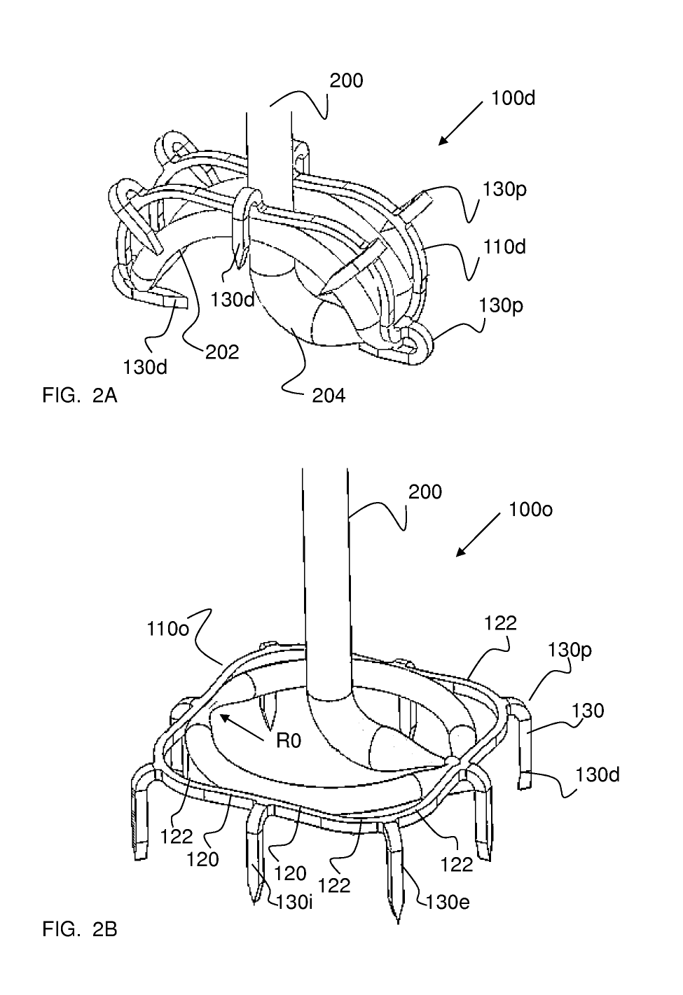

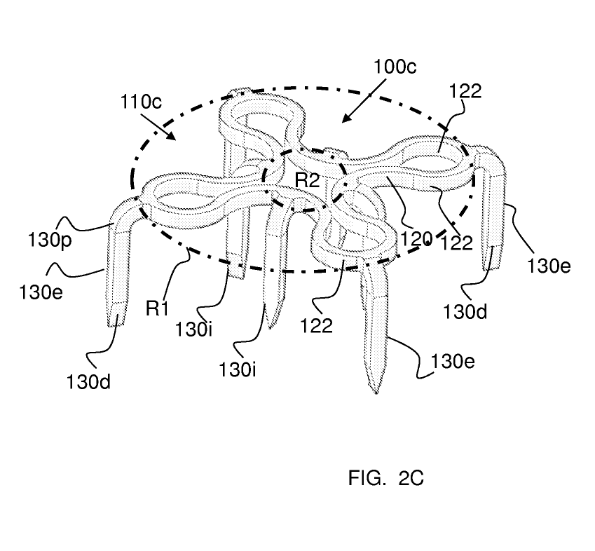

FIGS. 2A-C show schematic illustrations of an optional tissue closure device according to an optional embodiment of the present invention showing the different optional configurations of the tissue closure device 100 as utilized with an optional delivery tool 200. FIG. 2A shows the delivery and/or low profile and/or storage configuration 100d; FIG. 2B shows the open configuration 100o; FIG. 2C shows the closed configuration 100c.

Optionally and preferably closure device 100 may be provided from super-elastic and/or memory shape materials for example including but not limited to Ni—Ti alloys (nitinol), super-elastic titanium alloys, super-elastic metallic alloys, shape memory polymers, shape memory plastics, super-elastic titanium alloys, plastics and materials capable of super-elastic and/or memory shape properties and having a plurality of stable configurations.

Most preferably the different configuration of tissue closure device 100 may be changed and/or urged to assume the different configurations selected from delivery configuration 100d, open configuration 100o and closed configuration 100c. Optionally and preferably changing from one configuration to another may be provided for by applying a transition trigger optionally in the form of an environmental trigger and/or change for example including but not limited to temperature change. Optionally changing from one configuration to another may be provided for by a physical manipulation for example applying a mechanical force by manipulating the closure device with a tool. Optionally changing from one configuration to another may be provided by a combination of environmental trigger and a physical manipulation. Optionally and preferably the tissue closure device is configured to have a low profile transformation temperature of between −18° to 25° C. (degrees Celsius) and more preferably between 10° C. to 25° C. (degrees Celsius).

Optionally an environmental change may for example include but is not limited to temperature change, heat, cold, electrical field, electromagnetic field, magnetic field, chemical solution, the like or any combination thereof.

For example transforming closure device 100 into its low profile delivery configuration 100d, a non-limiting example of which is shown in FIG. 2A, may be provided by first cooling device 100 and thereafter applying a mechanical transformation, for example folding with a tool, to complete the transition into delivery configuration 100d of FIG. 2A. Similarly, transitioning to the open configuration 100o and/or closed configuration 100c may be provided for with the application and/or exposure to heat.

Most preferably tissue closure device 100 is configured to transition and/or transform from the delivery configuration 100d, FIG. 2A to open configuration FIG. 2B; and the open configuration may be transitioned and/or transformed to the closed configuration 100c, FIG. 2C.

FIG. 2A provides an illustrative depiction of the delivery and/or low profile, configuration 100d, of tissue closure device 100. The low profile and/or delivery configuration 100d is optionally and preferably associated with a carrying tool, applicator and/or delivery device 200. Optionally delivery device 200 may be provided in the form of a semi-compliant balloon shaped to receive tissue closure device 100 while allowing it to fold onto and/or while otherwise associated with device 200, as shown.

An optional semi-compliant delivery device 200, as shown, may comprise a ring-like closed loop portion 202 for associated with the closed loop surface 110 of closure device 100, and a shaft handle portion 204 for maneuvering closure device 100.

Optionally and preferably low profile delivery configuration 100d is provided by folding closed loop surface 110 onto itself optionally about a midline, and allowing anchors 130 to fold about anchor proximal end 130p to provide for a substantially planar device having tissue anchors 130 and closed loop surface 110 arranged in a coplanar manner.

FIGS. 2B and 2C depict the operative configuration, having an open configuration 100o, FIG. 2B, and closed configuration 100c, FIG. 2C, respectively. Optionally and preferably delivery configuration 100d is transition to open configuration 100o by application of and/or exposure to an environmental trigger such as heat and/or a physical manipulation. For example an environmental trigger may be provided in the form of exposure to body temperature to allow transition to the operative configuration from the delivery configuration. Optionally and preferably open configuration 100o maintains its shape with a central opening R0 with the assistance of delivery device 200. Optionally and preferably closed configuration 100c may be assumed once delivery device 200 is disassociated from closure device 100.

Optionally and most preferably the tissue closure device 100, assumes a delivery configuration 100d associated with delivery tool 200 that is adapted to be delivered to a tissue site 50 through an access point. At the tissue site 50 closure device 100d expands to assume open configuration 100o, while associated with delivery tool 200, such that closure device 100 has a diameter larger than the diameter of the access point. The tissue closure device 100, in the open configuration 100o, may associate with the tissue site opening 50 and allowed to assume a closed configuration 100c optionally and preferably by disassociating delivery tool 200, therein closing tissue site opening 50.

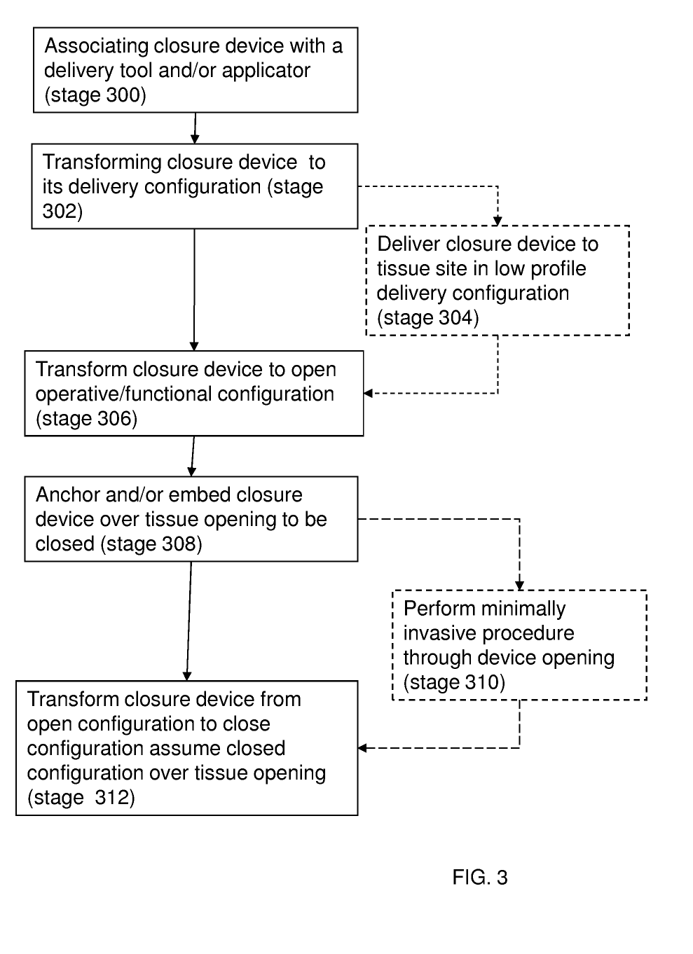

Referring now to FIG. 3, showing a flowchart of an optional method for tissue closure according to the present invention utilizing the tissue closure device 100 according to optional embodiments of the present invention.

First in stage 300, tissue closure device 100 according to optional embodiments of the present invention, is optionally and most preferably associated with an optional tool and/or delivery device 200 and/or applicator, for example provided in the form of a semi-compliant balloon 200, for example as shown in FIGS. 2A-B and 4A-E

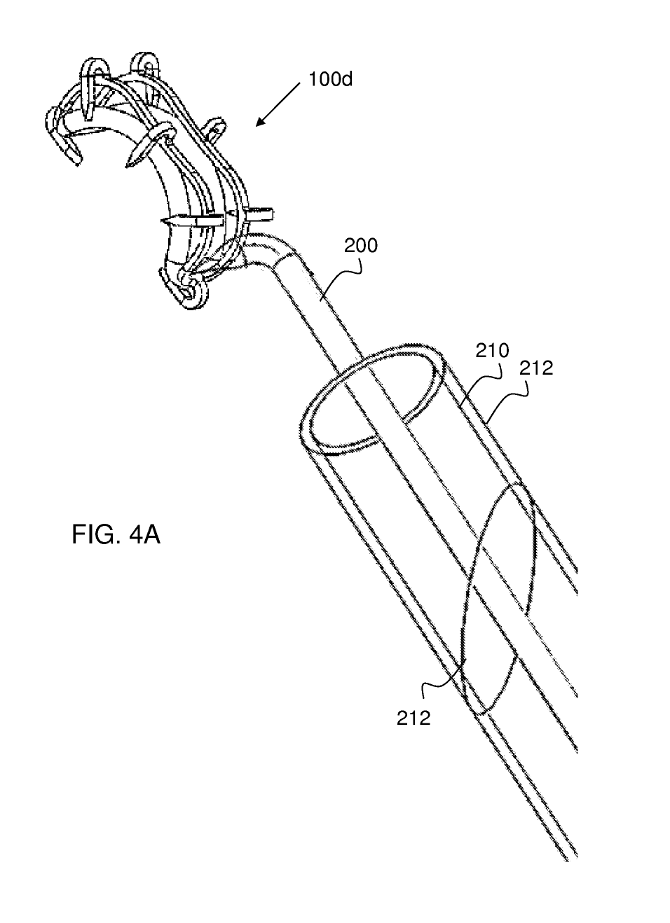

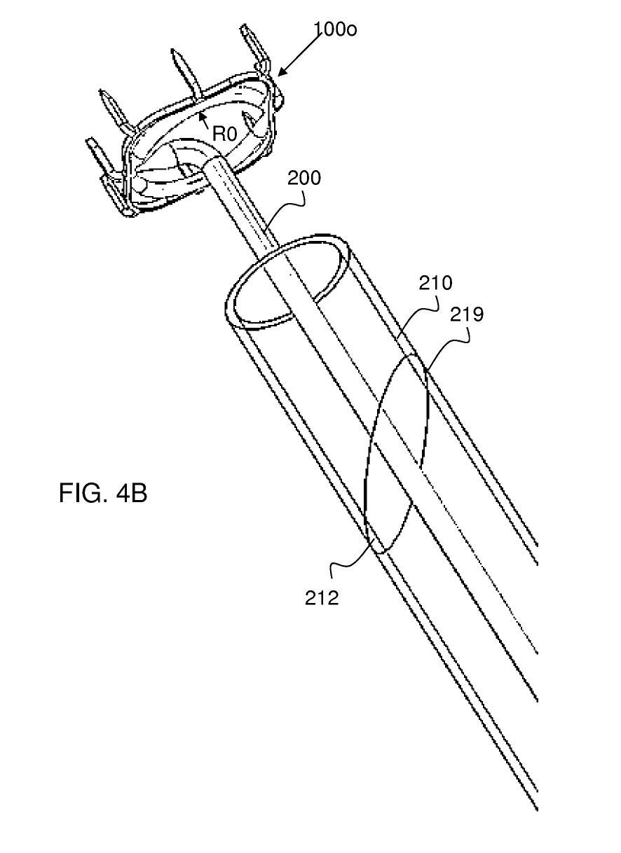

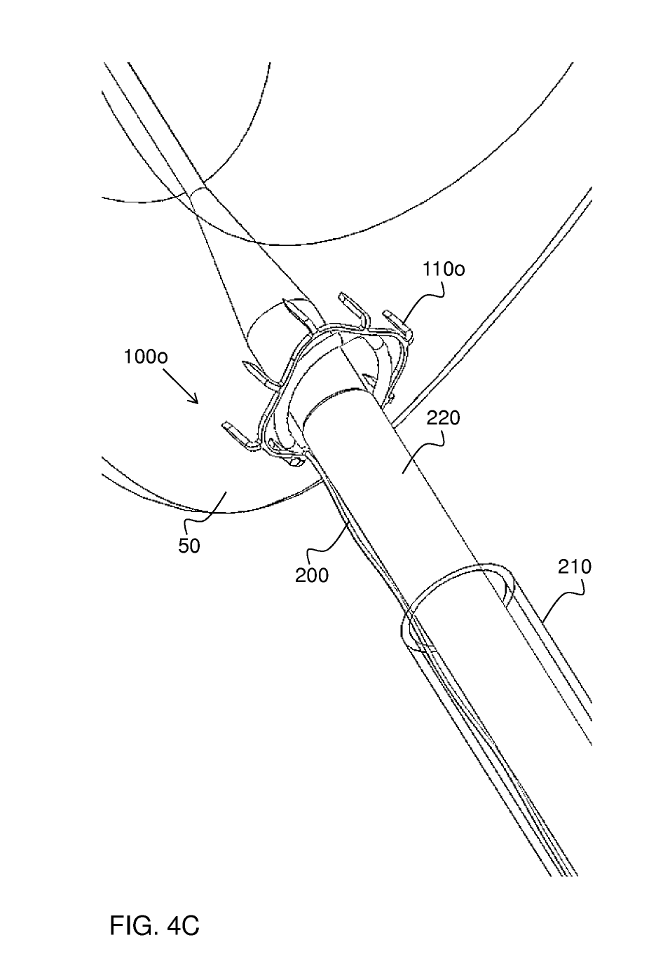

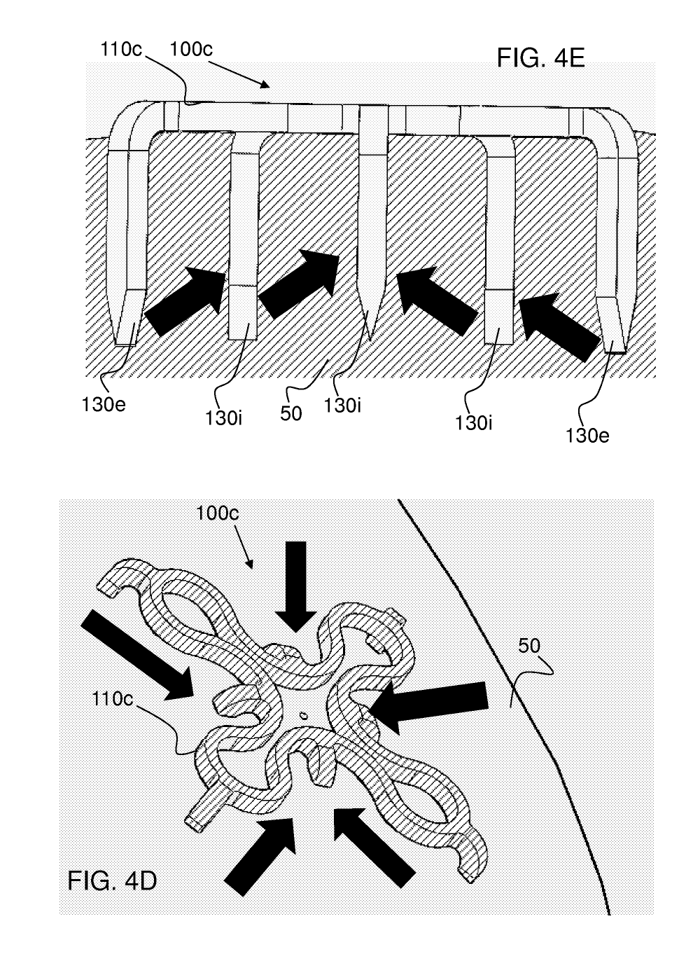

Next in stage 302 tissue closure device 100 is optionally and preferably transformed to its delivery configuration 100d. Optionally delivery configuration 100d may be provided by cooling closure device 100 and/or by mechanically folding device 100 to provide a low profile configuration 100d, FIG. 2A and FIG. 4A.

Optionally stages 300 and 302 may be undertaken during manufacturing and/or packaging of tissue anchor device 100, wherein tissue closure is provided in delivery ready form 100d and associated with a delivery device 200 and/or tool 200 and/or in a delivery catheter 210.

Next, in optional stage 304, most preferably required when delivery of tissue closure device 100 is provided as part of a minimally invasive procedure or surgery wherein tissue closure device 100 is introduced to tissue site 50 through a cannula, port, trocar or the like access point in the delivery configuration 100d for example via a delivery catheter 210 as described and shown in FIG. 4A-B. Most preferably tissue closure device 100 is advanced through catheter 210 toward a tissue site 50 and/or tissue wound and/or opening and/or access point that require closure.

Next in stage 306, tissue closure device 100 is prepared for deployment over the tissue site 50, most preferably by transitioning closure device from delivery configuration 100d to open configuration 100o. Optionally transformation to the open configuration 100o is triggered and/or provided with an environmental change in the form of heat exposure for example exposing closure device 100 to body temperature. Optionally other transformation triggers may be utilized as previously described, for example utilizing a tool to unravel closure device 100 from delivery formation 100d to open formation 100o.

Optionally, delivery device 200 may be provided in the form of an expanded semi-compliant balloon, provides for maintaining open configuration 100o by delaying and/or preventing transformation of closure device 100 to its closed configuration 100c.

Optionally and most preferably, the transformation from configuration 100d to 100o is facilitated by both a transformation trigger in the form of an environmental change, for example temperature change, and the use of an optional tool and/or delivery device 200 and/or applicator. For example, a temperature change, exposure to body temperature, may trigger and/or urge a transformation from 100d configuration to the closed configuration 100c of tissue closure device 100, while an associated tool and/or delivery device 200 and/or applicator may be utilized to at least partially counteract the full transformation from delivery configuration 100d to closed configuration 100c, allowing closure device 100 to assume the open configuration 100o defining an opening R0. Most preferably tissue closure device 100 is allowed to partially assume its closed configuration 100c at least with respect to a plurality of anchors 130, while closed loop surface 110 is maintained in the open formation 110o therein defining an opening R0 wherein tissue anchors 130 are exposed and ready for anchoring within the tissue site 50.

Next in stage 308, tissue closure device 100 in its open configuration 100o is anchored over the tissue site to be closed, by embedding a plurality of anchors 130 surrounding the open tissue site 50, wound, lesion, to be closed. Optionally and preferably tissue closer 100o is placed and or embedded such that anchors 130 surround open tissue site 50, wound, lesion, therefore the tissue site 50 is approximately centered within opening R0 defined by closure device 100o. Optionally tissue closure device 100o is embedded over at least a portion of tissue site 50.

Next, in optional stage 310, required when delivery and tissue closure is provide as part of a minimally invasive procedure, where the procedure is performed through opening R0. Optional stage 310, is necessary if it is desirable for tissue closure device 100 in its open configuration 100o to define the working space about the tissue, defined by opening R0, for example the myocardium, as shown and described with respect to FIG. 4A-E, therein allowing a minimally invasive procedure to be performed through the opening R0 defined by tissue closure device 100o.

Optionally, stage 310 may be omitted if placement of closure device 100 is required to close tissue lesions, wounds, holes or the like without defining a working area about the tissue, as shown and described in FIG. 4C

Finally in stage 312, once tissue closure device 100 in the open configuration 100o is in place (stage 308) and all tissue preparation and procedures are finalized such that the tissue site 50 may be closed over a tissue site opening, for example lesion, hole, wound or the like, tissue closure device 100o is optionally and preferably allowed and/or urged to assume its closed configuration 100c. Optionally allowing closure device 100o to assume the closed configuration 100c may be provided by removing any tool and/or delivery device 200 and/or applicator or the like associated therewith.

Optionally closure device 100o may be urged to actively transform into the closed configuration 100c by applying a transformation trigger for example in the form of an environmental change, as previously described.

Optionally closure device 100 may assume and/or transform to the closed configuration 100c directly from the delivery configuration 100d without an intermediate and/or intervening open configuration 100o, by exposing to a transition trigger for example temperature change.

Most preferably the closed configuration 100c defines the temperature stable configuration of closure device 100, while open configuration 100o defines an intermediate state that is preferably stabilized with a tool for example a delivery tool 200 in the form of a semi-compliant balloon 200 as described in FIG. 2B, 4B.

Referring now to FIGS. 4A-E showing schematic illustrations of an optional method for tissue closure of a tissue site 50 during a minimally invasive cardiac procedure, utilizing optional tissue closure device 100 shown in FIGS. 2A-C and the method described in FIG. 3.

FIG. 4A shows an optional embodiment for the delivery of closure device 100 in its low profile delivery configuration 100d (FIG. 2A) while the closure device 100 is associated with delivery device 200. Closure device 100d is delivered to a tissue site 50 with a delivery tool 200 for example through a delivery catheter 210 as previously described. Optionally and preferably delivery catheter 210 may be placed through a minimally invasive access point and/or port (not shown) or trocar 215, as shown. Most preferably the tissue closure device 100 may provide for closing a tissue site 50 having a diameter larger than the diameter of the access point and/or delivery catheter 210.

Initially, as previously describe in FIG. 2A and stage 302 of FIG. 3, tissue closure device 100 is configured to its low profile configuration 100d by triggering a transformation, with a transformation trigger for example including but not limited to an environmental change, exposure to cold and mechanical manipulation, for example folding about a midline.

Next, optionally and preferably closure device 100d may be loaded onto or placed through a trocar 215, port, cannula, delivery catheter 210, for example as shown in FIG. 4A and described in stage 304 of FIG. 3.

Next, as previously described in stage 306 and shown in FIG. 4B, delivery device 200 associated with closure device 100d is threaded toward the tissue site through the access point for example trocar 215 and/or catheter 210, as shown. Once reaching the tissue site 50 the tissue closure device 100, undergoes a transformation from its delivery configuration 100d to its operational and/or functional open configuration 100o defining an opening R0.

Next, as previously described in stage 310 and shown in FIG. 4C, once tissue closure device 100 assumes its operational and/or functional open configuration 100o, defining opening R0, device 100 may be anchored and/or embedded within the tissue, for example as shown about the tissue site 50 in the form of myocardium 50. Most preferably tissue closure device 100o is anchored onto tissue 50 by driving and/or anchoring anchors 130 to penetrate the tissue. Most preferably delivery device 200 is utilized to manipulate closure device 100o onto tissue 50 therein anchoring device 100 within the tissue by applying an embedding force.

Optionally the open formation of closed loop surface 110o provides for performing minimally invasive surgical manipulation for example with tools such as catheter 220, as shown in FIG. 4C, or the like tools through the opening R0 defined by closure device 100o such that the opening R0 is optionally and preferably maintained with delivery tool 200.

Finally, as previously described in stage 312 and shown in FIGS. 4D-E, once a procedure through the tissue 50 is finished the tissue site opening 50 may be closed by allowing tissue closure device 100 to assume and/or transform into its resting closed state 100c. To allow for tissue closure device 100 to transform to its functional and/or operation closed state 100c, delivery 200, optionally and preferably in the form of a semi-compliant balloon as previously described, is deflated allowing closed loop surface 110 to assume its closed configuration 110c, as shown, in FIG. 4D-E, over the tissue 50 opening, shown in the form of myocardium.

Direction arrows shown in FIGS. 4D-E depicts the closing force applied on the tissue surface 50 in order to close the tissue opening, Optionally and preferably such closing force is provided by the concentric configuration of the tissue anchors 130, 130e, and 130i where the relative force of transitioning closed loop surface 110 from its open formation 110o to the closed formation 110c forces tissue anchors 130 toward the center, in a concentric fashion where a plurality of peripheral and/or external tissue anchors 130e assume a first concentric radius and a plurality of inner tissue anchors assume a second concentric radius, therein forming a concentric closing force. The closing force is further optionally provided for by anchors 130 that optionally and preferably maintain their essentially perpendicular orientation relative to closed loop surface 110 during the transition form an open configuration 110o to a closed configuration 110c.

Referring now to FIGS. 2 and 5-7 depicting the structure of the tissue closure device 100 according to optional embodiments of the present invention, that most preferably provides sufficient closing force to close a tissue opening.

Most preferably tissue closure device 100 comprises a concentric configuration providing a stable and efficient closing force about a tissue opening. Optionally and preferably the concentric configuration further provide for creating a closing force gradient across the tissue site where the highest closing force is applied closest to the center of the tissue site 50, while the closing force applied reduces farther away from the center of the tissue site 50. Most preferably the applied closing force and/or closing force gradient provides for closing a tissue opening 50 having a diameter that is larger than the diameter of the access point itself.

Most preferably, tissue closure device 100 comprises a plurality of tissue anchors 130 extending from a closed loop surface 110.

Most preferably tissue anchors 130 are disposed on a plane at an angle that is essentially perpendicular and/or orthogonal with respect to the plane of the closed loop surface 110.

Optionally tissue anchors 130 may be provided with a thickness of about 0.5 mm. Optionally tissue anchors 130 are provided with a length of about 5 mm to about 7 mm. Optionally and preferably tissue anchors are provide with a length of about 6 mm. Most preferably tissue anchors 130 are configured to have a length extending from closed loop surface 110 from about 5 mm to about 7 mm.

Optionally closed loop surface 110 or any portions thereof provided from nitinol, may be configured to have a thickness for example from about 0.25 mm to about 2.0 mm, optionally from about 0.5 mm to about 1.75 mm, optionally and preferably from about 0.7 mm to about 1.6 mm, and most preferably about 1.5 mm.

Optionally and most preferably anchors 130 may be arranged about the perimeter of surface 110 in such a manner so that the closed configuration 110c comprises tissue anchors 130 arranged in a concentric manner, for example as shown in FIG. 2C, 5A-B, 6A-B, defining at least two or more concentric radii R1, R2, R3.

Most preferably tissue anchors 130 comprise a proximal end 130p and a distal end 130d. Optionally and preferably the distal end provides for anchoring/embedding device 100 around tissue 50 to be closed. Optionally and preferably tissue anchor proximal end 130p may be fluid with and extends from closed loop surface 110, as shown in FIG. 2A-C.

Optionally and preferably the orientation of anchors 130 relative to closed loop surface 110 may be configured to have a low profile and/or folded and/or delivery configuration, as shown in FIG. 2A, or an erect and/or functional configuration, as shown in FIGS. 2B-C.

Optionally and preferably the different tissue anchor 130 configuration, for example including but not limited to erect and/or functional and/or low profile and/or folded and/or delivery configurations are adapted and controlled about the tissue anchor's proximal end 130p.

Optionally and preferably the low profile delivery configuration of anchors 130 is provided such that proximal end 130p may be folded allowing anchor 130 to assume a coplanar position with respect to closed loop surface 110. Optionally and preferably tissue anchors 130 in its functional configuration, wherein proximal end 130p extends and assumes a plane essentially perpendicular with respect to the plane of closed loop surface 110.

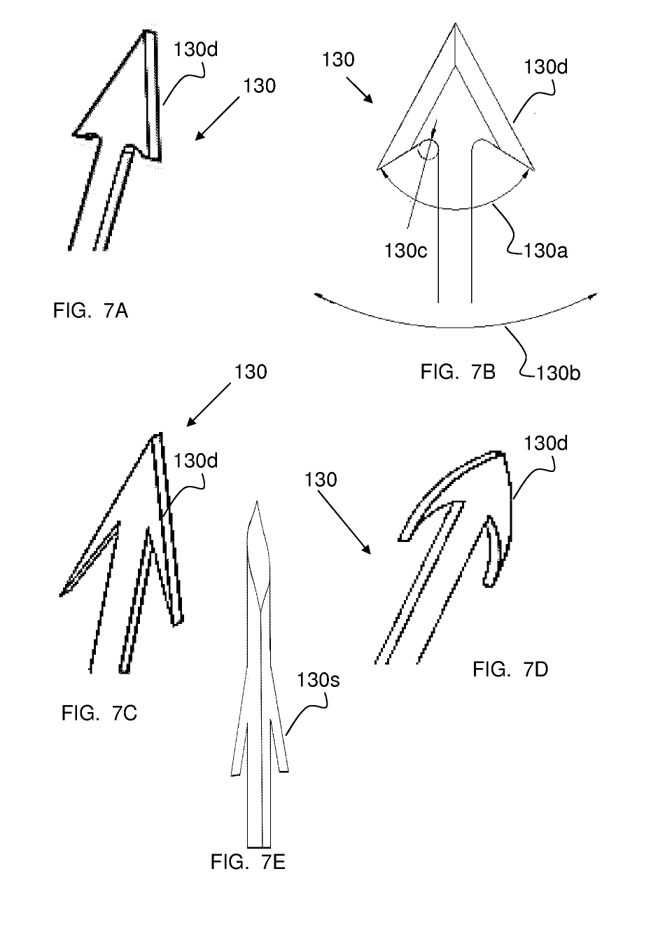

Optionally and preferably tissue anchor distal end 130d define anchoring projection configured for tissue anchoring and providing an embedding force of up to about 60 g, utilized to anchor and/or embed device 100 within target tissue 50. Tissue anchor distal end 130d may optionally be configured to have varying shapes adapted for anchoring tissue closure device 100 within the tissue, for example including but not limited to barb, hook, multi-barb, serration, threading, anchor, multi-prong anchor, arrowhead, skyved projection, the like or any combination thereof, for example as show in FIGS. 7A-E.

Optionally and preferably anchors 130 may be configured to be anchored in place and not readily removed once placed over the tissue site to be closed.

Optionally individual anchors 130 are configured and/or adapted to exert a holding force from about 60 g to about 180 g, providing for anchoring and maintaining tissue closure device 100 within the tissue. Optionally and preferably the holding force provided may be from about 80 g to about 100 g. Most preferably the holding force is only realized and/or exerted when a counterforce attempting to eject or remove device 100 from the tissue site 50. For example a tissue anchor 100 disposed on the myocardium may utilize the holding force to ensure that the beating myocardium does not eject closure device 100 due to its natural activity within the cardiac cycle. Optionally and preferably the holding force exerted by a plurality of tissue anchors depend and/or correlate to anchors' 130 respective and relative positions within the tissue site 50 and the characteristics of the tissue site 50 itself.

Optionally anchors 130 may be adapted and/or customized to provide a holding force according to the tissue type to be closed.

Optionally, individual tissue anchors 130 may be adapted to have individual and/or varying holding force configuration. Optionally, tissue anchors 130 are adapted to have a uniform holding force.

Optionally the holding force of anchors 130 may be adapted based on the location of anchors 130 about closed loop surface 110, within the external zone R1 or internal zone R2, FIG. 2C. For example, internal anchors 130i, may be adapted to have a higher holding force than external anchors 130e. For example, internal anchors 130i, may be adapted to have a lower holding force than external anchors 130e.

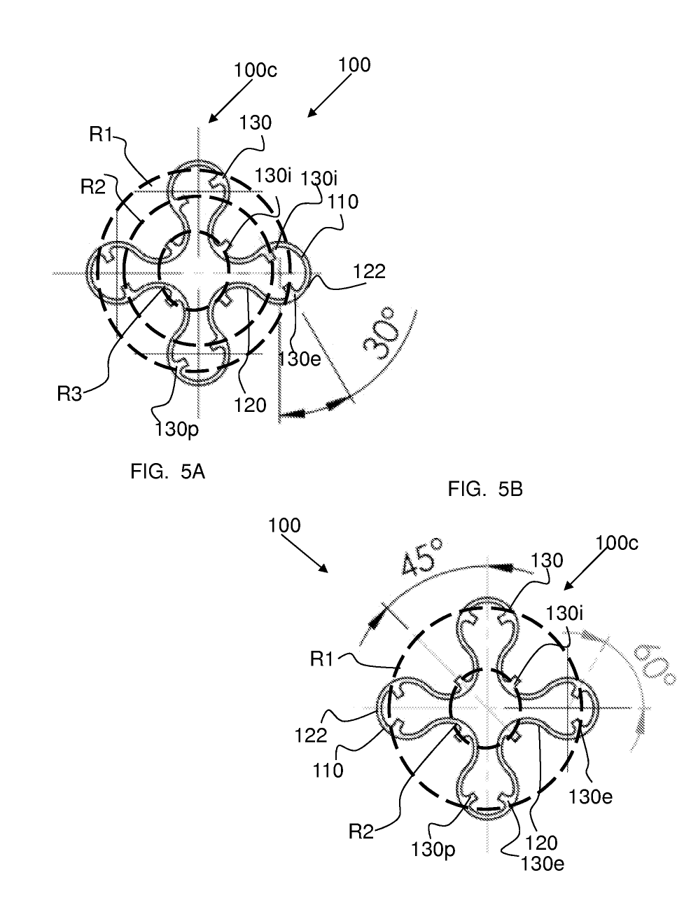

Optionally tissue anchors 130 may be adapted to have different properties about their proximal end 130p and/or distal end 130d. For example, a tissue closure device 100 may have different types of tissue anchors 130, for example including but not limited to internal tissue anchor 130i and peripheral and/or external tissue anchors 130e. Optionally internal tissue anchors 130i may be adapted to apply a first closing force while external tissue anchors 130e may be adapted to apply a second closing force. Optionally internal tissue anchors 130i may be adapted to provided a plurality of closing forces depending on which concentric radii they are disposed R2, R3, therein providing a closing force gradient about tissue opening 50. For example an internal tissue anchors 130i disposed about R2 will have a first internal closing force while internal tissue anchor 130i disposed about R3 will have a second internal closing force. Most preferably the second internal closing force is higher than the first internal closing force. Optionally and preferably closing force about the internal anchors is highest at the innermost tissue anchor disposed about the innermost concentric radius, for example R3 shown in FIG. 5A, 6B-C.

Most preferably closed loop surface 110 may be configured to have a plurality of configurations including: a low profile and/or delivery configuration 110d, (FIG. 2A) an open configuration 110o (FIG. 2B) and a closed configuration 110c (FIG. 2C).

Optionally and preferably delivery configuration 110d defining a low profile of the device, most preferably adapted for storage or delivery to a tissue site 50 through an access point, trocar, cannula, port, catheter or the like.

Most preferably closed loop surface 110 may comprise at least two functional and/or operative configurations including an open configuration 110o (FIG. 2B) and closed configuration 110c (FIG. 2C).

Optionally and preferably open configuration 110o, FIG. 2B, may assume any closed loop surface shape for example including but not limited to circular, oval, elliptical, polygon, or the like geometric shape defining a closed loop surface.

Optionally and preferably surface 110o provides an opening R0 defined by a diameter from about 16 mm to about 25 mm, more preferably from about 19 mm to about 20 mm. Therefore, most preferably open configuration 110o provides for closing a tissue opening 50 having a diameter that is larger than the diameter of the tissue opening defined by the access port.

Most preferably a plurality of anchors 130 may define internal anchors 130i arranged about a first concentric radius for example R1 defining at least one internal zone R1, shown in FIG. 2B; while a plurality of anchors 130 may define peripheral and/or external anchors 130e, arranged about a second concentric radius R2, shown in FIG. 2B-C defining an external and/or peripheral zone and/or concentric radius R1.

Optionally and preferably peripheral zone and/or concentric radius R1 comprises a diameter that is smaller than the diameter of the opening of tissue closure device in its open configuration 110o. Optionally peripheral zone R1 a diameter from about 13 mm to about 19 mm.

Optionally and preferably at least one internal zone and/or concentric radius R2 may be configured to have a diameter smaller than the diameter of the peripheral zone R1. Optionally internal zone R2 comprises a diameter from about 7 mm to about 13 mm. Optionally internal zone R2 comprises a diameter from about 9 mm to about 12 mm. More preferably internal zone R2 comprises a diameter of about 10 mm.

Most preferably transitioning to the closed configuration 110c provides for generating a closing force a force from about 200 g to about 500 g on the tissue to be closed, more preferably a force of about 350 g. Optionally and preferably, closing force is generated between the peripheral zone R1 and the internal zone R2. Most preferably the concentric configuration provides for sustaining the closing forces about the center of the tissue site 50, wound, lesion to be closed.

Optionally closed loop surface 110 may be provided from a continuous closed loop surface comprising a plurality of segments, 120 and 122. Optionally individual segments 120, 122 may be associated with at least one or more tissue anchor 130.

Optionally individual segments 120, 122 may be associated with either an internal zone anchor 130i or a peripheral zone anchor 130e.

Optionally, closed loop surface 110 may be a continuous loop comprising a plurality of segments 122, 120 wherein individual segments 120, 122 corresponds to either an internal zone anchor 130i or a peripheral zone anchor 130e.

For example, segment 120 may optionally be adapted and/or configured to form a portion of the closed loop surfaced 110 corresponding to and/or forming an integral part of internal concentric zone for example R2, while segment 122 may optionally be adapted and/or configured to form a portion of the closed loop surfaced 110 corresponding to and/or forming an integral part of external concentric zone for example R1.

Optionally, closed loop surface 110 may be formed from a continuous surface comprising a plurality of concatenated segments, 122, 120 wherein each segment 120, 122 is selected from an internal zone surface segment or a peripheral zone surface segment.

Optionally closed loop surface 110 may be customized, in terms of the number and sequence of segments 120, 122 utilized to construct and/or form closed loop surface 110. Optionally, the customization may be provided based on the tissue type to be closed. Optionally closed loop surface 110 may be constructed by alternating internal zone surface segment 120 and the peripheral zone surface segment 122 to form a close loop configuration 110.

Optionally the tissue anchors 130 may be shaped in an arrow-head like and/or barb configuration, about the distal end of the anchors. Preferably tissue anchor shape provides for securely anchoring and/or embedding tissue closure device within a given tissue. Tissue anchor distal end 130d provided in the form of an arrow-head may be configured to have a width of from about 1 mm to about 4 mm, more preferably about 2 mm.

An optional tissue anchor distal end 130d provided in the form of an arrow-head may be configured to have an internal angle, optionally internal angle is an obtuse angle, for example from about 100 degrees to about 125 degrees, more preferably form about 110 to about 120, and most preferably about 115 degrees.

An optional tissue anchor distal end 130d provided in the form of an arrow-head may be configured to have an external angle, optionally the external angle is an acute angel, for example from about 40 degrees to about 75 degrees, optionally and preferably from about 45 to about 65, more preferably form about 50 to about 60, and most preferably about 56 degrees.

An optional tissue anchor distal end provided in the form of an arrowhead may be configured to have an internal angle formed between the tissue anchor's central shaft and arrow-head having a diameter of about 0.15 mm.

Optionally and preferably each segments forming the closed loop surface portion of tissue closure device according to the present invention is provided with a margins of about 0.7 mm. Optionally and preferably a 0.7 mm buffer provides for maintain material properties such as flexibility.

FIGS. 5A-B shows schematic illustrations of optional tissue closure device according to optional embodiments of the present invention. FIGS. 5A-B show tissue closure device 100 in its closed configuration 100c having optional tissue anchor configurations about closed loop surface 110.

Most preferably tissue anchors 130 may be distributed about the perimeter of the closed loop surface 110, uniformly or non-uniformly about the perimeter of closed surface 110. Optionally tissue anchor 130 distribution may be based on their location within at least one or more internal zones R2/R3 or external zone R1.

For example, as shown in FIG. 5A, anchors 130 forming the innermost internal zone R3 are dispersed at a 45 degree angle relative to the center and arranged at 90 degrees from an adjacent anchor 130i. Anchors 130 forming internal zone R2 are arranged to be 180 degrees from anchors 130 forming external zone R1, that may be arranged at 30 degrees, as shown. Accordingly, while all tissue anchors are arranged about the perimeter of close surface 110 their placement and/or arrangement about surface 110 determines the number of concentric zones provided to closure device 100. Most preferably a plurality of concentric zones provides for improved tissue closure by exerting sufficient force to close the tissue wound, incision, opening, most preferably by applying a radial closing force exerted toward the center of tissue closure device 100.

FIG. 5B shows a further optional tissue anchor 130 arrangement about surface 110, forming two concentric zones R1 external and an internal zone R2. Internal zone R2 anchors are distributed about a 45 degree angle as described above. However, external zone anchors R1 are arranged to be 120 degrees from one another therein forming external zone R1.

An optional embodiment of the present invention provides for placing tissue anchors 130 about the perimeter of surface 110 so as to configure the required amount concentric zones. Optionally the number of concentric zones may be determined based on or in proportion to at least one or more parameters for example including but not limited to tissue type, the force required to close the tissue, the size of the tissue opening, any combination thereof or the like.

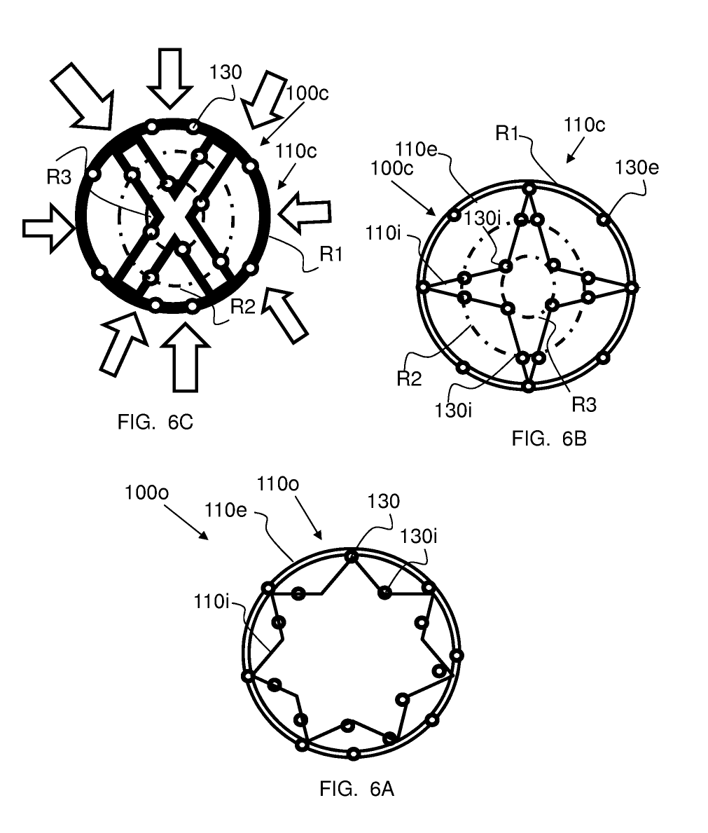

FIG. 6A-C show an optional illustrative schematic diagrams of an optional tissue closure device according to the present invention comprising a concentric configuration.

Optionally the closed loop surface may be constructed from at least two closed loop surfaces configured in concentric manner, for example as shown in FIG. 6A-C. Optionally the tissue closure device may be configured to have a first closed loop surface, for example an outer closed loop structure R1, and an internal closed loop R2, R3.

Optionally at least two closed loop concentric structures may be combined to form the tissue closure device 100.

FIG. 6A-B show optional closed configuration 100c of tissue closure device 100, according to an optional embodiment of the present invention. Tissue closure device 100c, as shown comprise a plurality of tissue anchors 130 arranged in a concentric fashion, providing an optional 3 ring concentric ring configuration where the tissue anchors are dispersed concentrically about 3 different radii, a first concentric radius R1, a second concentric radius R2 and a third concentric radius R3 all having a common center, most preferably to ensure tissue closure with sufficient closing force toward the common center defined by the tissue 50 lesion, opening, wound, hole being closed. Most preferably each concentric radius may be associated with at least one and more preferably a plurality of tissue anchors 130. Optionally and preferably, concentric radius R1 defines a radius comprising peripheral and/or external anchors 130e while concentric radii R2 and R3 define the radii comprising internal anchors 130i.

Optionally closure device may be configured to have at least two and more preferably a plurality of concentric arrangements.

FIG. 6C shows a schematic illustration of a tissue closure device 100 in an open configuration 100o that may for example be transformed to a closed configuration 100c as shown in FIG. 6A-B. Tissue closure device 100o optionally comprises at least two closed loop surface an outer closed loop surface 110e and an inner closed loop surface 110i. Optionally and preferably each of outer surface 110e and inner surface 110i comprise a plurality of tissue anchors 130, dispersed thereabout. Optionally inner surface 110i may comprise inner tissue anchors 130i that most preferably form the inner concentric radii, for example about R2 as shown in FIG. 6A-B.

Optionally tissue closure device 100 may be transformed between the open configuration 100o, for example as shown in FIG. 6C, to a closed configuration 100c, for example as shown in FIG. 6A-B, with a transformation trigger and/or with a tool for manipulating closure device 100. Optionally a transformation trigger my for example include but is not limited to an environmental change, temperature change, application of heat, application of cold, electrical field, electromagnetic field, magnetic field, chemical solution, the like or any combination thereof.

Optionally and preferably during such a transformation and/or transition between open configuration 100o and closed configuration 100c, inner closed surface 110i would transition to assume a closed configuration 110c from an open configuration 110o therein applying a closing force. Optionally and preferably the closing force is resultant from the transformation of outer closed loop surface 110e and inner closed loop surface 110i from their respective open configuration (FIG. 6C) to their respective closed configuration (FIG. 6A-B), therein pulling and/or forcing tissue anchors 130 to move toward the center of tissue opening, wound, lesion, incision, in concentric and/or radial direction. Optionally and preferably the closing force, shown with directional arrows FIG. 6A, is applied about a plurality of concentric radii, for example R2 and R3.

FIGS. 7A-E are schematic illustrations of optional distal end 130d of tissue anchors 130 of tissue closure device 100 according to the present invention. FIGS. 7A-E show optional configuration of the distal end 130d of tissue anchors 130, in the form of arrowhead anchors.

The arrowhead configuration disposed bout the distal end 130d provides for coupling and/or anchoring and/or embedding tissue closure device 100 at a tissue site 50 having a wound, lesion and/or opening to be closed.

Optionally the configuration of anchors distal end 130 for example in the form of an arrowhead, as shown FIGS. 7A-E, may be configured according to at least one or more parameters for example including but not limited to internal angle 130a, external angle 130b, shaft-head interface angle, shaft-head interface radius 130c, shaft length, anchor length, anchor width, anchor thickness, edge configuration, edge sharpness, edge angle, any combination thereof or the like.

Referring to FIG. 7B shows an optional tissue anchor distal end 130d provided in the form of an arrow-head comprising a shaft-head interface radius 130c formed between the tissue anchor central shaft and arrow-head shaped distal end 130d, where the interface radius 130c may be about 0.15 mm. Optionally tissue anchor distal end 130d may be configured to have an internal angle 130a of about 115 degrees, as shown, and external angle of about 56 degrees.

As shown with respect to FIG. 7B, optionally the arrowhead width may be configured to have a width of about 2 mm.

Optionally and preferably the tissue anchors may be provided with a thickness of about 0.5 mm. Optionally tissue anchors are provided with a length of about 5 mm to about 7 mm. Optionally and preferably tissue anchors are provide with a length of about 6 mm.

Optionally and preferably tissue anchor distal end 130d define an anchoring projection configured for tissue anchoring and providing an embedding force. Optionally embedding force may be configured relative to tissue type and/or tissue status. Optionally, in healthy cardiac tissue, tissue anchors may be configured to have an embedding force of up to about 60 g.

Optionally and preferably arrowhead distal end 130d, FIG. 7A-D as shown, are configured to be anchored in place and not readily removed once placed over the tissue site to be closed.

Optional distal end 130d provided with skyved projections 130s such as that shown in FIG. 7E, may most preferably be utilized to exert a holding force that may be realized when a counterforce attempting to eject or remove device 100 from the tissue site 50, such that skyved projection 130s would expand to anchor device 100 within tissue 50 in the presence of an ejection force. Optionally tissue anchor 130 may comprise at least one or more skyved side projection 130s that may extend from the sides of anchor' body. Most preferably at least one or more skyved projection 130s may provide for easy insertion into a tissue site 50, minimizing peripheral bleeding, while adequately preventing removal from the tissue site 50, as the skyved projection 130s protrudes and/or extend from anchor 130 body with a removal attempt and/or force. Optionally and most preferably skyved projection 130s provide a holding force for the tissue anchor; therein preventing extraction from the tissue site 50. Optionally and most preferably skyved projection 130s extends from the body of anchor 130 with the exertion of a perpendicular force on the anchor. Optionally and preferably skyved tissue anchor's, FIG. 7E, may be provided from SMA or SA for example including NiTi or the like alloys.

Optionally anchors 130 are configured and/or adapted to exert a holding force from about 60 g to about 180 g, providing for anchoring and maintaining tissue closure device 100 within the tissue site 50. Optionally and preferably the holding force provided may be from about 80 g to about 100 g.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

Having described a specific preferred embodiment of the invention with reference to the accompanying drawings, it will be appreciated that the present invention is not limited to that precise embodiment and that various changes and modifications can be effected therein by one of ordinary skill in the art without departing from the scope or spirit of the invention defined by the appended claims.

Further modifications of the invention will also occur to persons skilled in the art and all such are deemed to fall within the spirit and scope of the invention as defined by the appended claims.

While the invention has been described with respect to a limited number of embodiments, it will be appreciated that many variations, modifications and other applications of the invention may be made.

1. A tissue closure device provided from shape memory or super-elastic materials for closing a tissue site opening, the device comprising a plurality of tissue anchors extending from a closed loop surface wherein the plane of said tissue anchors is provided at an angle essentially perpendicular with respect to the plane of said closed loop surface;
a. wherein said plurality of tissue anchors comprise a proximal end and a distal end; said distal end provided for anchoring said device onto tissue; and wherein said proximal end is fluid with and extends from said closed loop surface; and
b. said closed loop surface having a plurality of configurations including: a delivery configuration, an open configuration and a closed configuration wherein said closed loop surface may undergo a transformation between one of said plurality of configurations to another; said delivery configuration defining a low profile of said device and adapted for delivery to a tissue site through a small profile access point having a first diameter and wherein said closure device is utilized to close a tissue site having a second diameter such that said second diameter is larger than said first diameter.

2. The device of claim 1 wherein said plurality of tissue anchors comprise a plurality of configurations including: a delivery configuration and at least one functional configuration.

3. The device of claim 1 wherein said open configuration is provided with a diameter from about 16 mm to about 25 mm.

4. The device of claim 1 wherein said closed configuration is provided with a concentric arrangement comprising an external zone and at least one internal zone.

5. The device of claim 4 wherein said external zone is provided with a diameter from about 13 mm to about 19 mm.

6. The device of claim 4 wherein said at least one internal zone is provided with a diameter from about 7 mm to about 13 mm.

7. The device of claim 4 configured to generate a closing force between said external zone and said internal zone, said closing force is configured to close said tissue site opening by exerting a radial force from about 200 g to about 500 g.

8. The device of claim 1 configured to apply a closing force gradient centered about said tissue site opening, wherein the closing force is highest nearest to the center of said tissue site opening and lowest at the periphery of said tissue site opening.

9-10. (canceled)

11. The device of claim 1 wherein said transformation is effected with an environmental change.

12. The device of claim 11 wherein said environmental changes includes temperature change.

13. The device of claim 1 wherein said tissue anchors are adapted to have a length from about 5 mm to about 7 mm.

14. The device of claim 1 wherein individual tissue anchors are adapted to exert a holding force from about 60 g to about 180 g.

15. The device of claim 14 wherein the level of said holding force may be adapted by manipulating or configuring the distal end of said tissue anchors.

16. The device of claim 1 wherein said tissue anchors are adapted to individually apply an embedding force of less than about 60 g.

17. The device of claim 1 wherein said delivery configuration provides for delivery facilitated by a low profile delivery device or tool.

18. (canceled)

19. The device of claim 1 wherein said open configuration defines a closure device central opening that provides for performing minimally invasive surgical manipulation therethrough.

20. The tissue anchors of claim 1 wherein said distal end define anchoring projection configured for tissue anchoring and providing an embedding force, said projection selected from the group consisting of barb, hook, multi-barb, serration, threading, anchor, multi-prong anchor, arrow head, skyved projection anchors.

21. The device of claim 1 wherein said shape memory or super-elastic materials are provided with material thickness from about 0.7 mm to about 1.6 mm.

22. A method for closing a tissue site opening with a tissue closure device according to claim 1, the method comprising:
a. associating said tissue closure device with a delivery tool or applicator; and
b. transforming said tissue closure device to a delivery configuration;
c. delivering said closure device in said delivery configuration to a target tissue opening to be closed;
d. transform said tissue closure device from said delivery configuration to an functional open configuration, therein defining a closure device central opening, sufficient to encompass and close said targeted tissue site opening;
e. anchor said tissue closure device about target tissue site opening with the aid of said delivery tool; and
f. Transform said closure device from said functional open configuration to a functional closed configuration therein closing said tissue site opening.

23. The method of claim 22 wherein, delivering said tissue closure device comprises maneuvering said closure device in its delivery configuration, through a small profile access point selected from the group consisting of trocar, cannula, keyhole, and catheter.
